(19) United States
(12) Patent Application Publication
(10) Pub. No.: US 2011/0270137 A1
(43) Pub. Date: Nov. 3, 2011

(54) METHOD AND SYSTEM FOR TREATING SKIN TISSUE

(75) Inventors: Alon GOREN, Moshav Ben-Shemen (IL); Michael Kardosh, Kiryat-Ono (IL)

(73) Assignee: Applisonix Ltd., Rechovot (IL)

(21) Appl. No.: 13/096,037

(22) Filed: Apr. 28, 2011

Related U.S. Application Data
(60) Provisional application No. 61/329,144, filed on Apr. 29, 2010.

(51) Int. Cl. A61N 7/00

(52) U.S. Cl. 601/2

(57) ABSTRACT

A device for treating a skin tissue is disclosed. The device comprises a device body having an anterior compartment and being formed with at least one suction port, and an ultrasound unit configured for generating an ultrasound wave into the anterior compartment. When an under pressure is formed in the suction port, the skin tissue is drawn by vacuum into the anterior compartment and irradiated by the ultrasound wave to form a focal spot optionally and preferably at a distance of from about 0.2 mm to about 4 mm below an external surface of the skin tissue.

### RELATED APPLICATION

This application claims the benefit of priority under 35 USC §119(e) of U.S. Provisional Patent Application No. 61/329,144 filed Apr. 29, 2010, the contents of which are incorporated herein by reference in their entirety as if fully set forth herein.

### FIELD AND BACKGROUND OF THE INVENTION

The present invention, in some embodiments thereof, relates to tissue treatment and, more particularly, but not exclusively, to a method and system for treating skin tissue.

The skin or integument is a major organ of the body present as a specialized boundary lamina, covering essentially the entire external surface of the body, except for the mucosal surfaces. Structurally, the skin organ is complex and highly specialized as is evidenced by its ability to provide a barrier against microbial invasion and dehydration, regulate thermal exchange, act as a complex sensory surface, and provide for wound healing wherein the epidermis responds by regeneration and the underlying dermis responds by repair (inflammation, proliferation, and remodeling), among a variety of other essential functions.

Known in the art are several techniques for treating skin and other anatomical tissue by applying ultrasound power. For example, U.S. Pat. No. 6,936,046 describes skin rejuvenation by thermal stimulation using high intensity focused ultrasound (HIFU). Ultrasound power is applied perpendicular to the skin and within the body.

U.S. Pat. No. 6,113,559 discloses the use of ultrasound therapy for reducing rhytides of the skin. A low power and low frequency focused ultrasound beam is applied to the skin in order to stimulate or irritate the dermis layer to cause a change in the dermis layer that results in a change in the smoothness of the epidermis. The ultrasound is applied for period of time sufficient to cause synthesis and/or production of new connective tissue that results in reduction or elimination of human skin wrinkles. The ultrasound is applied perpendicular to the skin and within the body.

U.S. Pat. No. 6,500,141 teaches an apparatus for treating soft surface tissue with ultrasound. Ultrasound is irradiated from an applicator surface facing the body surface from outside through the body surface into the body tissue. A suction apparatus generates sucking in the body surface against the applicator surface. The treatment is applied to the epidermis, dermis and fat tissue layers. The ultrasound energy is focused by means of the inward curvature of the applicator surface. Several mechanisms for preventing heating are disclosed.

U.S. Pat. No. 5,665,053 discloses an endermology body massager adapted to simultaneously apply ultrasound for treating cellulite while massaging a tissue. Two rollers spaced from each other in a parallel configuration rotate in the same direction. A vacuum source is connected to the chamber that houses the rollers.

International Publication No. WO2007/102161 discloses monitoring and applying of ultrasound to the skin, for cosmetic treatments, including body contouring by lipolysis, hair removal, wrinkle and face lift, or face-localized molding of adipose tissue. The tissue is sucked into a housing, and ultrasound energy is transmitted typically parallel to the skin of the subject. The ultrasound energy is designated to remain in large part within the housing and tissue therein, and generally not to affect tissue outside of the housing. The ultrasound transducers are arranged in a circle such that ultrasound energy transmitted by the transducers remains generally within a plane defined by the circle. The duration of sonication is sufficiently long to enable lypolisys.

### SUMMARY OF THE INVENTION

According to an aspect of some embodiments of the present invention there is provided a device for treating a skin tissue. The device comprises a device body having an anterior compartment and being formed with at least one suction port, and an ultrasound unit configured for generating an ultrasound wave into the anterior compartment.

According to some embodiments of the invention the device body and the ultrasound unit are designed and constructed such that when an under pressure is formed in the suction port, the skin tissue is drawn by vacuum into the anterior compartment and being irradiated by the ultrasound wave to form a focal spot at a distance of from about 0.2 mm to about 4 mm below an external surface of the skin tissue.

According to some embodiments of the invention the ultrasound unit comprises an annular ultrasound transducer and wherein at least part of the device body is defined by the annular ultrasound transducer.

According to some embodiments of the invention the annular ultrasound transducer has a shape selected from the group consisting of a ring, a truncated cone and a spherical segment.

According to some embodiments of the invention the ultrasound unit comprises a plurality of elongated linear ultrasound transducers configured to generate ultrasound waves in a plurality of directions being generally parallel to an external skin surface outside the anterior compartment and adjacent thereto.

According to some embodiments of the invention the ultrasound unit comprises a pair of elongated linear ultrasound transducers configured to generate ultrasound waves in opposite and co-linear directions being generally parallel to an external skin surface outside the anterior compartment and adjacent thereto.

According to some embodiments of the invention the ultrasound unit comprises an ultrasound transducer generally shaped as a half-cylinder, wherein the suction port(s) is formed at a wall of the transducer generally along an apex of the half-cylinder.

According to some embodiments of the invention the acoustic unit comprises an acoustic waveguide defining the anterior compartment and an ultrasound transducer being acoustically coupled to the acoustic waveguide such that the acoustic wave is transmitted from the ultrasound transducer to the and from the acoustic waveguide to the skin tissue.

According to some embodiments of the invention the acoustic waveguide comprises a pair of contact lips opposite to each other, the contact lips being configured for contacting the skin tissue such that ultrasound energy is delivered to the skin tissue via the contact lips but not via any other part of the device.

According to some embodiments of the invention a distance between the contact lips is selected such as to form a standing wave therebetween.

According to some embodiments of the invention the acoustic waveguide has a wavy structure having ridges and troughs and wherein the anterior compartment is defined within the troughs.

According to some embodiments of the invention the acoustic waveguide is formed with at least one pipe therein having a first end at a trough of the wavy structure and a second end at a wall of the wavy structure to form the suction port(s).

According to some embodiments of the invention the acoustic unit comprises a plurality of cylindrical ultrasound transducers, and wherein the anterior compartment is defined between two adjacent transducers.

According to some embodiments of the invention the cylindrical ultrasound transducers are arranged within the device body, wherein the suction port(s) is formed at a wall of the device body.

According to some embodiments of the invention the cylindrical ultrasound transducers are operative to roll on a surface of the skin tissue.

According to some embodiments of the invention the acoustic unit comprises an ultrasound transducer having an ultrasound emitting surface which is slanted at an angle with respect to an external skin surface outside the anterior compartment and adjacent thereto.

According to some embodiments of the invention the device further comprises an ultrasound matching layer at least partially occupying the anterior compartment and being selected for enhancing acoustic coupling between the ultrasound wave and the skin tissue.

According to some embodiments of the invention the ultrasound matching layer is a gel being in direct contact with an internal wall of the anterior compartment.

According to some embodiments of the invention the ultrasound matching layer is self-supporting layer.

According to some embodiments of the invention the device further comprising a top member at which the suction port is formed, wherein the top member is characterized ultrasound reflectivity of less that 50%.

According to some embodiments of the invention the device further comprising a suction device being an fluid communication with the suction port and configured for generating the under pressure.

According to some embodiments of the invention the device further comprises a cooling conduit configured for redirecting air sucked from the anterior compartment onto the acoustic unit so as to cool the acoustic unit.

According to some embodiments of the invention the device further comprises a controller configured for controlling and/or monitoring at least one parameter, selected from the group consisting of time of treatment, frequency of the ultrasound wave, power of the ultrasound wave, intensity the ultrasound wave, vacuum level within the anterior compartment, temperature of the skin tissue, and depth of the focal spot.

According to some embodiments of the invention the controller is configured for controlling the vacuum level so as to adjust the depth of the focal spot.

According to some embodiments of the invention the device further comprises at least one sensor for providing the controller with data pertaining to at least one of the parameters.

According to some embodiments of the invention the device further comprises a feeding device configured for feeing the anterior compartment with an acoustic matching material.

According to some embodiments of the invention the controller is configured for controlling the feeding device so as to select a volume of the acoustic matching material hence to control a free volume within the anterior compartment hence also to adjust a depth of the focal spot.

According to some embodiments of the invention the controller is configured for accessing a pain-level database and comparing at least one the parameters to parameters in the pain-level database so as to assess a pain level associated with the skin tissue treatment.

According to some embodiments of the invention the device further comprises a pay-per-use interface communicating with the controller.

According to an aspect of some embodiments of the present invention there is provided a method of treating a skin tissue. The method comprises drawing the skin tissue by vacuum into an anterior compartment of a device body and generating an ultrasound wave into the anterior compartment, such as to form a focal spot at a distance of from about 0.2 mm to about 4 mm below an external surface of the skin tissue.

According to some embodiments of the invention the focal spot is formed in the dermis of the skin tissue but not in the epidermis thereof.

According to some embodiments of the invention the focal spot is formed in the dermis of the skin tissue but not in the hypodermis thereof.

According to some embodiments of the invention the ultrasound wave is generated along a direction being generally parallel to an external skin surface outside the anterior compartment and adjacent thereto.

According to some embodiments of the invention the ultrasound wave is generated in a direction which is slanted at an angle with respect to an external skin surface outside the anterior compartment and adjacent thereto.

According to some embodiments of the invention the ultrasound wave is transmitted only to the sides of the drawn skin tissue.

According to some embodiments of the invention the ultrasound wave is generated by a plurality of rotatable cylindrical ultrasound transducers, wherein the method comprises rolling the cylindrical ultrasound transducers on the surface of the skin tissue.

According to some embodiments of the invention the method further comprises reducing reflection of the ultrasound wave from a top part of the anterior compartment.

According to some embodiments of the invention the method further comprises adjusting vacuum level in the anterior compartment so as to adjust the depth of the focal spot.

According to some embodiments of the invention the method further comprises feeding acoustic matching material into the anterior compartment, wherein the an amount of the matching material is selected so as to adjust a depth of the focal spot.

According to some embodiments of the invention the method further comprises assessing a pain level associated with the skin tissue treatment.

According to some embodiments of the invention the treating comprises at least one treatment selected from the group consisting of: increasing collagen growth, hair removal, hair root destruction, wound cleaning, wound sanitizing, destruction of acne, acupuncture points stimulation, blood vessels treatment, tattoo removal, infection prevention, fibroblast cells stimulation, increasing the production of adenosine triphosphate (ATP), sweat glands treatment, skin cleansing, massaging, increasing local blood flow, microdermabrasion, and pressure sores treatment.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a typical structure of a skin;

FIGS. 2A-E are schematic illustrations of a device suitable for treating skin tissue, according to some embodiments of the present invention;

FIG. 3 is a schematic illustration of the relation between the direction of the ultrasound wave and the external surface of the epidermis;

FIGS. 4A-E show the effect of a top member of a device according to some embodiments of the present invention on the location of the focal spot;

FIG. 5 is a schematic illustration of an ultrasound transducer in embodiments of the invention in which the transducer is shaped as a ring with a slanted internal surface;

FIGS. 6A-B are schematic illustrations of ultrasound transducers in embodiments of the invention in which the transducers comprise a plurality of elongated linear transducers;

FIG. 7 is a schematic illustration of an ultrasound transducer in embodiment of the invention in which the transducer is shaped as half a cylinder;

FIG. 8 is a schematic illustration of a device suitable for treating skin tissue, in embodiments of the present invention in which the device comprises one or more sensors;

FIG. 9 shows a Wong-Baker face rating scale which can be used according to some embodiments of the present invention;

FIG. 10 is a schematic illustration of a device suitable for treating skin tissue in embodiments in which the device comprises an acoustic waveguide designed and constructed for delivering acoustic energy the sides to the treated tissue;

FIG. 11 is a schematic illustrations of a device suitable for treating skin tissue in embodiments in which the device comprises an acoustic waveguide having a wavy structure;

FIG. 12 is a schematic illustration of another wavy structure, according to some embodiments of the present invention;

FIG. 13 is a schematic illustration of a device suitable for treating skin tissue in embodiments of the invention in which cylindrical transducers are employed; and

FIG. 14 is a schematic illustration of a device suitable for removing hair or at least partially destroying hair roots, according to some embodiments of the present invention.

### DESCRIPTION OF SPECIFIC EMBODIMENTS OF THE INVENTION

The present invention, in some embodiments thereof, relates to tissue treatment and, more particularly, but not exclusively, to a method and system for treating skin tissue.

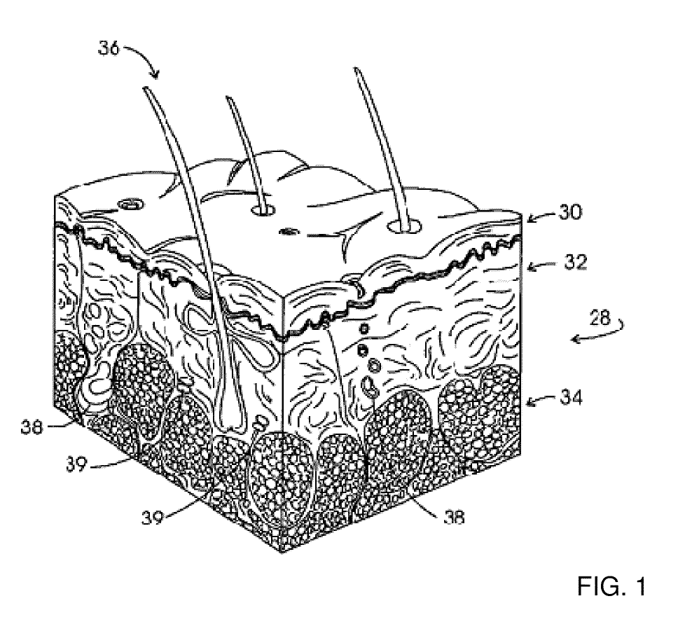

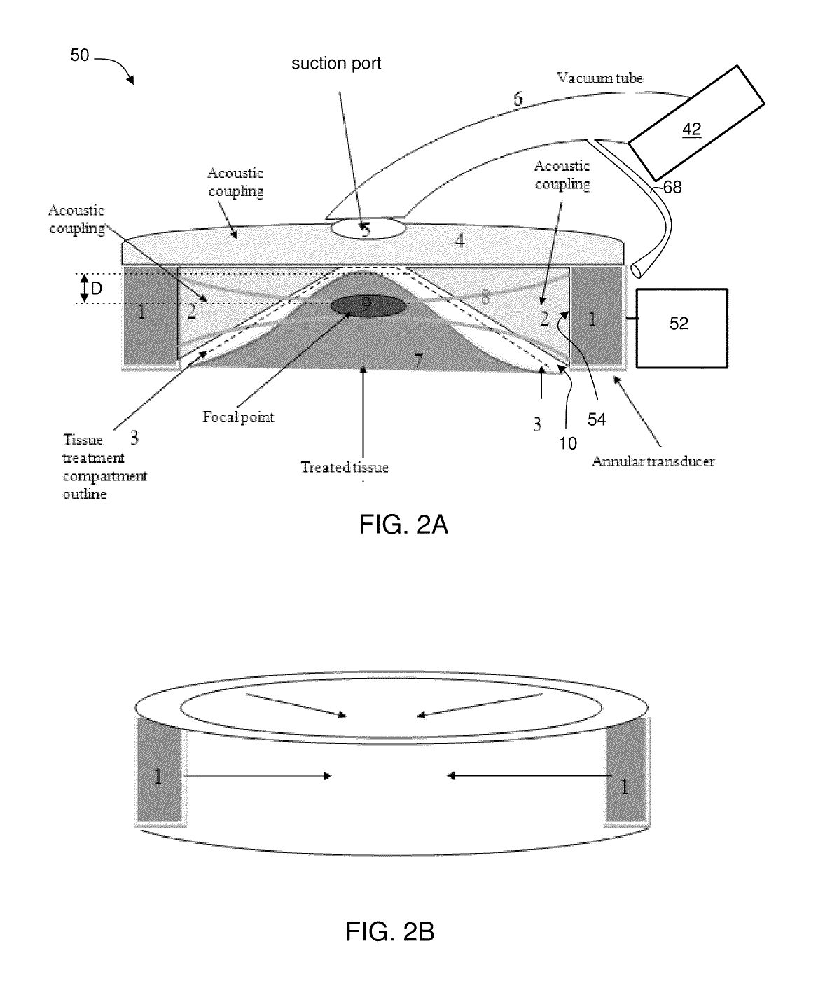

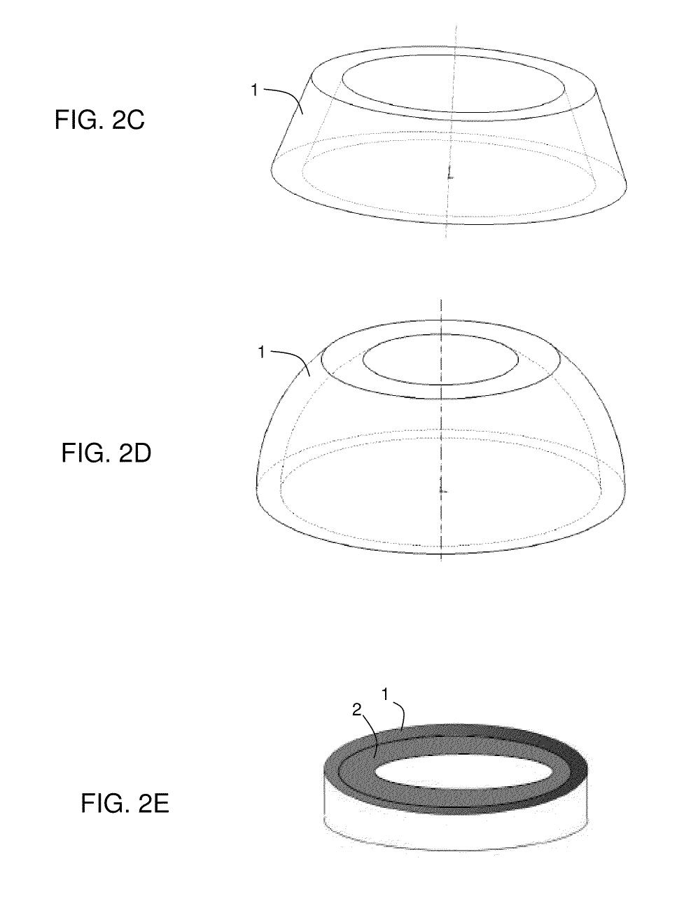

For purposes of better understanding some embodiments of the present invention, as illustrated in FIGS. 2-14 of the drawings, reference is first made to the typical structure of the skin as illustrated in FIG. 1.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

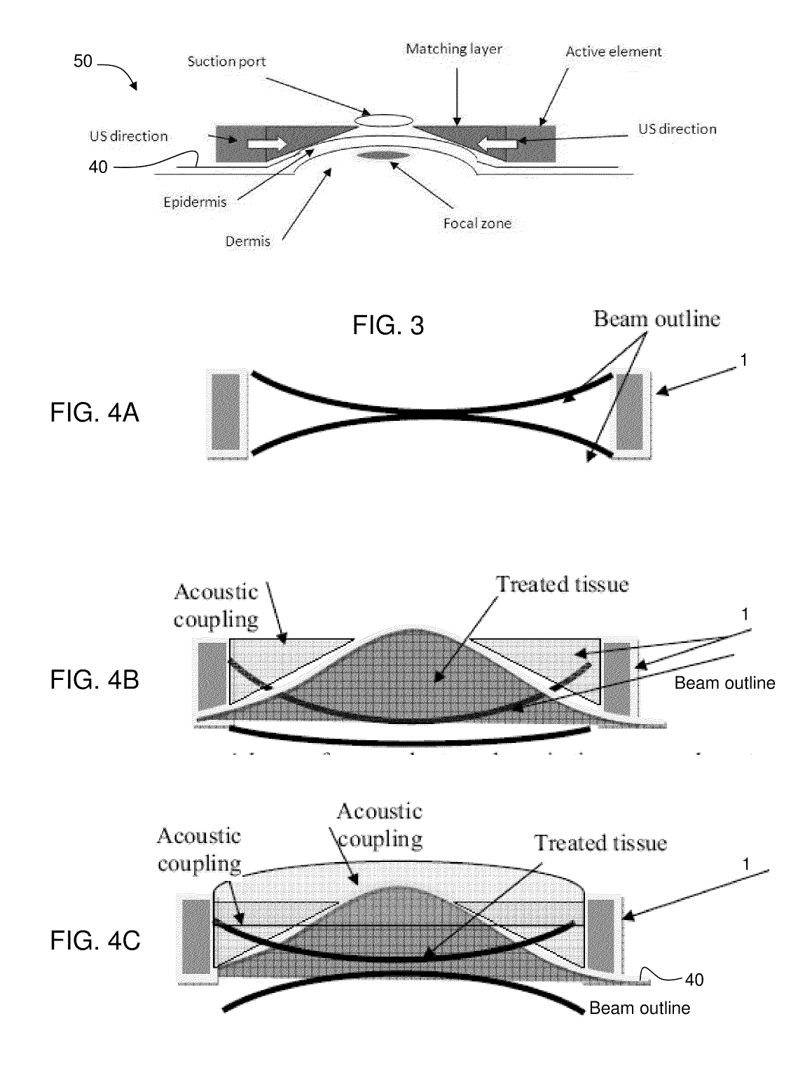

FIG. 1 is a schematic illustration showing a typical structure of the skin 28. The illustrated skin structure is one of two major skin classes of structure and functional properties representing thin, hairy (hirsute) skin which constitutes the great majority of the body's covering. This is as opposed to thick hairless (glabrous) skin from the surfaces of palms of hands, soles of feet and the like. In the figure, the outer epidermis layer 30 is shown generally extending over the dermis layer 32. Dermis 32, in turn, completes the integument and is situated over an adjacent subcutaneous tissue layer 34, also known as the hypodermis. Additionally shown in FIG. 1 are the hair follicle and an associated shaft of hair 36, vascular structures 39 feeding dermis 32 and sweat glands 38. Not shown in FIG. 3 are a number of other components, including the cellular structure of the dermis, and the vascular tissues supplying the vascularized dermis and its overlying epidermis.

Epidermis 30 in general comprises an outer or surface layer, stratum corneum composed of flattened, cornified non-nucleated cells. This surface layer overlays a granular layer, stratum granulosum composed of flattened granular cells which, in turn, overlays a spinous layer, stratum spinosum composed of flattened polyhedral cells with short processes or spines and, finally, a basal layer, stratum basale, composed columnar cells arranged perpendicularly. The epidermis typically consists of between 30 to 50 cell layers and is about 200 μm thick. Generally, the epidermis contains no blood vessels.

Dermis 32 in general comprises a papillary layer, subadjacent to the epidermis, and supplying mechanical support and metabolic maintenance of the overlying epidermis. The papillary layer of the dermis is shaped into a number of papillae that interdigitate with the basal layer of the epidermis, with the cells being densely interwoven with collagen fibers. The reticular layer of the dermis merges from the papillary layer, and possesses bundles of interlacing collagen fibers that are typically thicker than those in the papillary layer, forming a strong, deformable three dimensional lattice around the cells of the reticular dermis. In addition to the collagen, which contributes to the firmness of the skin, the dermis also includes elastin which imparts flexibility and durability. The potential outcome of increased collagen levels in facial skin is a reduction of wrinkles with enhanced skin resilience and a more youthful appearance. Generally, the dermis is highly vascularized, especially as compared to the avascular epidermis. The dermis also includes sebaceous glands and nerves. The dermis exhibits a thickness of from about 1.0 mm to about 4.0 mm.

Hypodermis 34 consists primarily of loose connective tissue and lobules of fat, and it contains larger blood vessels and nerves than those found in dermis 32. The hypodermis exhibits a thickness which varies from person to person and also from one body area to the next. The hypodermis in women is typically thicker than in men.

The aftermath of soft tissue injury involves three phases of tissue repair. A good estimate of the time required to complete the complete injury repair process is 60 to 90 days. As individuals age the body's ability to repair injury or trauma is diminished. Collagen production and function diminish dramatically in aging skin with an estimate of 1% total collagen loss for each year of life over 20 years. Younger skin exhibits a much more organized and orderly arrangement of dermal collagen and elastin fibers.

Table 1, below depicts the average depth of the epidermis and the dermis layers. In most areas of the body the epidermis is only 35 to 50 microns thick. In the area around the eye it is only about 20 microns thick.

TABLE 1
Average depth of the epidermis and the dermis layers

| Layer | Range of thickness (mm) | Average (mm) |
|---|---|---|
| Epidermis | 0.07-1.4 | 0.735 |
| Dermis | 0.6-3.0 | 1.8 |

When ultrasound energy is applied to a tissue, significant physiological effects may be produced in the tissue resulting from thermal and/or mechanical and/or bio-chemical and bio-cellular effects changes or effects in the tissue.

The thermal effect, which is caused due to ultrasound absorption, includes heating of the tissue. When the tissue is heated to a sufficiently high temperature, tissue damage such as coagulative necrosis is produced. Within the dermis, heat causes contraction and thickening of the collagen fibers. It wad found by the present inventors that this change facilitates support overlying tissues and results in an overall tightened appearance of the skin. Heat within the dermis creates a limited thermal injury. The body's natural response to this injury is to produce collagen at the site of the wound. This results in firmer, thicker, more youthful skin. As the collagen-rich dermis is heated above 60° C., it denatures, and the fibrils contract and thicken. In the months following treatment, the body's natural wound healing process generates new collagen and dermal remodeling, leaving the skin looking firmer and more youthful.

The remodeling system in the dermis is a function of both the temperature to which the dermis is raised and the duration of time that the dermis is kept at an elevated temperature. The relationship between time and temperature can be plotted on a curve of equal effect, derived from the Arrhenius' rate equation [Ross, et al., Arch Dermatol (1999); 134(4): 444].

Reference is now made to FIGS. 2A-E which are schematic illustrations of a device 50 suitable for treating skin tissue, according to some embodiments of the present invention. In various exemplary embodiments of the invention device 50 is a Vacuum ENhanced Ultrasound (VENUS) device, which simultaneously applies ultrasound radiation and vacuum to the tissue.

Device 50 is particularly useful for treating dermal tissue, e.g., dermal layers below the epidermis or dermis boundary. Device 50 comprises one or more ultrasound transducers 1, which provides the ultrasound radiation. Typically, transducer 1 vibrates at an ultrasonic frequency of from about 0.5 MHz to about 10 MHz. Also contemplated, are embodiments in which a combination of several frequencies is employed in order to better focus the ultrasound at the desired spot. In various exemplary embodiments of the invention the frequency is a resonant frequency which characterizes the dimension of the internal cavity of device 50. In some embodiments of the present invention the frequency is varied during operation (for example, by performing a frequency sweep, such as up-chirp or down-chirp) so as to vary the ultrasound near filed extent. The acoustic power at the surface 54 of transducer 1 is preferably from about 1 W/cm² to about 5 W/cm².

Transducer 1 can be of any type. Representative examples including, without limitation, a piezo ceramic transducer, a solid transducer, a piezo film transducer and the like.

In some embodiments of the present invention transducer 1 has an annular shape, e.g., a ring, a truncated, a spherical segment or the like. A typical internal diameter of the ring is from about 10 mm to about 15 mm, and a typical height of the ring is from about 1 mm to about 3 mm. A typical wall thickness of the ring is from about 0.5 mm to about 2 mm. A view of transducer 1 according to some embodiments of the present invention is schematically illustrated in FIG. 2B. As shown, the ultrasound points inward with respect to the ring. Although FIG. 2B illustrates transducer 1 as a ring, this need not necessarily be the case, since, for some applications, it may not be necessary for the transducer to have a shape of a ring. For example, transducer 1 can be shaped as a truncated cone, as schematically illustrated in FIG. 2C, or a spherical segment, as illustrated in FIG. 2D. Other shapes are not excluded from the scope of the present invention. Additional exemplary transducers are provided hereinunder.

Optionally and preferably device 50 further comprises a tissue treatment compartment (TTC) 10 into which the tissue 7 to be treated is preferably sucked by vacuum. The outline of tissue treatment compartment 10 is shown at 3. When transducer 1 is shaped as a ring or ring, at least part of tissue treatment compartment 10 is preferably within the anterior of the transducer 1. The volume of tissue treatment compartment 10 is partially occupied by a matching layer 2, configured for enhancing the acoustic coupling between the ultrasound radiation and tissue 7. A view of transducer 1 (for the non-limiting case of ring-like transducer) with matching layer 2 according to some embodiments of the present invention is schematically illustrated in FIG. 2E.

Matching layer 2 can be made of any material suitable for enhancing the acoustic coupling with the tissue. Representative examples include, without limitation, water, ultrasonic coupling gel, soft ultrasonic transparent gels or cushions, PVC, polyurethane and the like. Matching layer 2 can be provided in a gel form directly applied onto the internal surface of transducer 1 or the tissue to be treated, or it can be provided as a self-supporting layer, for example, a gel enclosed in an ultrasound transparent balloon (e.g., a latex rubber balloon).

The top member 4 of compartment 10 is optionally and preferably has a curvature with its concave surface facing the anterior of the compartment. In some embodiments of the present invention member 4 is shaped as a dome. Member 4 serves for reducing boundary effects within compartment 10 so as to generate a focal spot at a predetermined location.

As used herein “focal spot” refers to a region in which the ultrasound intensity reaches a local maximum by means of constructive interference. The region can be of any dimension, namely a zero-dimensional region (a focal point), a one-dimensional region (a focal line), a two-dimensional region (a focal surface) or a three-dimensional region (a focal volume).

Member 4 is preferably designed and constituted so as not to reflect ultrasound radiation. In some embodiments of the present invention member 4 is characterized by ultrasound reflectivity of less than 50%. This can be achieved, for example, by providing a dome made of a material characterized by ultrasonic properties which are similar to the ultrasonic properties of tissue 7. A representative example for a material suitable for the present embodiments includes, without limitation, a silicone rubber, e.g., room-temperature vulcanizing (RTV) silicone rubber and the like.

Within member 4, there is one or more suction ports 5 connectable to a vacuum tube 6 so as to allow suctioning of air from compartment 10 thereby to generate under pressure in compartment 10. Vacuum tube 6 can be connected to a suction device 42 for generating an under pressure in tube 6.

When device 50 is placed on the skin and vacuum is applied through tube 6, tissue 7 is partially sucked into compartment 10. Once tissue 7 is within compartment 10, ultrasound transducer is activated to form an ultrasound beam 8 within compartment 10. Beam 8 forms a focal spot 9 within a predetermined depth in tissue 7. Ultrasound reflection from the surfaces of transducer 1 and/or the boundary of tissue 7 can also serve to buildup the focal spot. Also contemplated, are embodiments in which tissue 7 is positioned at the focal spots of the near field of transducer 1. In this configuration, two or more treatment zones are formed at the same time. Optionally and preferably, at least a portion of the air that is sucked from the anterior of compartment 10 is redirected, for example, by means of a cooling conduit 68, and released onto the external part of transducer 1 so as to cool the transducer.

There is more than one advantage in the generation of vacuum within compartment 10.

One advantage is that is allows emission of the ultrasound wave towards a predetermined depth within tissue 7, and reduces or prevents penetration of ultrasound radiation into deeper tissues. Another advantage is that the vacuum stretches the epidermis thus reducing the acoustic absorbance and, therefore, the heat dissipated in it. More specifically, the vacuum increases the surface area of the skin and reduces the epidermis thickness. As a result, the amount of energy transmitted through the skin to the targeted area at the dermis is increased and the heat absorbed in the epidermis is reduced. An additional advantage is that it affects mesenchymal cells known as fibroblasts. Fibroblasts primarily perform the repair and maintenance of connective tissues. The activity of these cells is regulated, in part, by changes in the mechanical environment in which they resides. Mechanical stress (e.g., vacuum) stimulates the fibroblasts and leads to collagenesis (see, for example, Fibroblasts responses to mechanical forces, Proceedings of the Institution of Mechanical Engineers, Part H: Journal of Engineering in Medicine 1998; 212(2):85-92 Eastwood M McGrouther D a, Brown R A).

An additional advantage of the vacuum suction is that the vacuum level is controllable. Such control can be utilized to select the volume of tissue within compartment 10. This is advantageous since it allows controlling the depth of the ultrasound focal spot, wherein larger tissue volume within compartment 10 generally to deeper location of the focal spot.

In various exemplary embodiments of the invention the focal point is generated at dermal layers immediately below the epidermis or at the boundary between the dermis and epidermis. Typical depth of the focal point below the skin surface is from about 0.2 mm to about 4 mm, more preferably from about 0.5 mm to about 4 mm. In various exemplary embodiments of the invention there are no focal spots at a depth which is less than about 0.2 mm, and in various exemplary embodiments of the invention there are no focal spots at a depth which is more than about 4 mm.

Transducer 1 can be activated either in a continuous mode or in pulsed mode as desired. In various exemplary embodiments of the invention the direction of ultrasound radiation is such that the ultrasound wave is predominantly tangential to the surface of tissue 7. FIG. 3 schematically illustrates the relation between the direction of the ultrasound wave and the external surface of the epidermis, according to some embodiments of the present invention. As shown, the ultrasound wave is generally parallel (e.g., within ±10° deviation) to an external skin surface 40 outside the anterior of compartment 10 and adjacent thereto

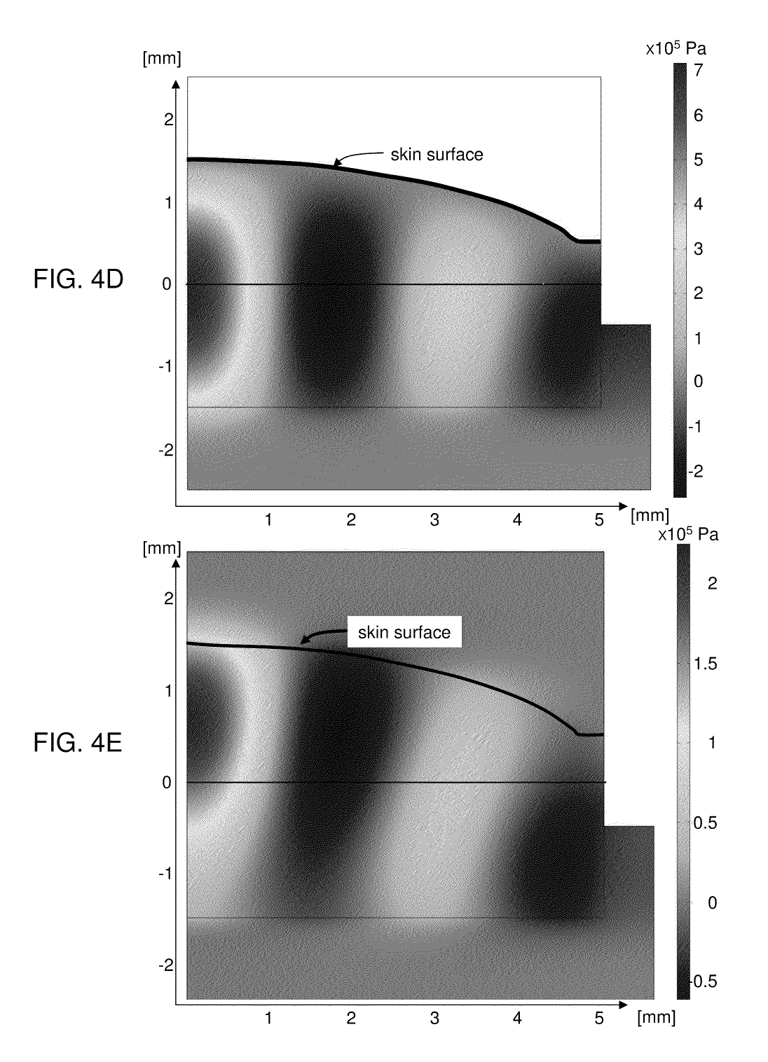

The effect of member 4 on the location of the focal spot can be better understood with reference to FIGS. 4A-E. FIG. 4A illustrates the ultrasound beam generated by an ultrasound transducer 1 shaped as a ring, in the absence of tissue within the compartment (e.g., when transducer 1 is filled with uniform liquid). For clarity of presentation, only the outline of the beam is illustrated. As shown, the beam is symmetric, with a focal spot on the symmetry axis of the ring. FIG. 4B illustrates the ultrasound beam in the presence of tissue 7 and matching layer 2. Tissue 7 introduces boundary effects which break the symmetry of the ultrasound wave and shifts the focal spot deep into the tissue.

FIG. 4C illustrates the ultrasound beam in the presence of tissue 7, matching layer 2 as well as top member 4. As shown, member 4 generally restores the symmetry of the ultrasound wave and shifts the focal spot towards the surface of the skin.

FIGS. 4D and 4E show results of computer simulations performed in accordance with some embodiments of the present invention to investigate the effect of member 4 on the location of the focal spot. Shown in FIGS. 4D and 4E are ultrasound pressure maps visualized using a color code. A color bar representing the color code is shown on the right-hand-side of each map.

The simulations were performed for a device which included a transducer shaped as a ring (inner diameter 10 mm, height 3 mm) operating at a frequency 500 kHz. FIG. 4D shows the results of simulation in which the ring was open at both sides, and FIG. 4E shows the results of simulation in which the ring was capped with a top member shaped as a dome and having acoustics properties (density and speed of sound) which are the same as those at the anterior of the ring.

In FIGS. 4D and 4E, the vertical axis correspond to coordinates along the symmetry axis of the ring and the horizontal axis correspond the radial coordinate, where the origin is defined at the geometrical center of the ring. The surface of the skin is illustrated as a thick black line. The curvature of the surface indicates the suction operation into the ring. As demonstrated in FIGS. 4D and 4E, the top member successfully shifts the focal spot (dark red region) closer to the surface of the skin. Specifically, in the absence of the top member (FIG. 4D) the center of the generated focal spot is approximately 1.5 mm below the surface of the skin, whereas in the presence of the top member (FIG. 4E) the center of the generated focal spot is approximately 0.8 mm below the skin.

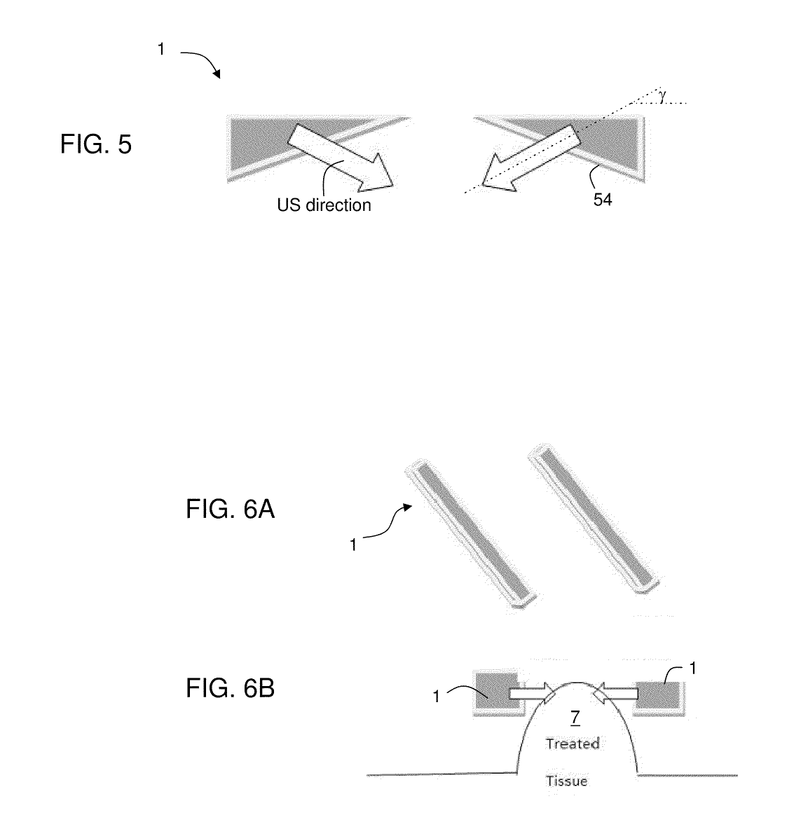

FIG. 5 is a schematic illustration of transducer 1 in embodiments of the invention in which transducer 1 is shaped as a ring with a slanted internal surface 54. For example, the outer surface of transducer 1 can be cylindrical and the inner surface 54 can be truncated conical. The surface is slanted at an angle γ with respect to the horizontal direction (or, equivalently, an angle 90−γ with respect to the symmetry axis of the ring). A typical value of γ is from about 0° to about 60°. The angle γ affects the direction of the ultrasound wave and therefore also the vertical location of the focal spot. Thus, γ is preferably selected so as to form the focal spot at a predetermined depth below the skin surface or a predetermined range of depths below the skin surface.

FIGS. 6A and 6B are schematic illustrations of transducers 1 in embodiments of the invention in which transducers 1 comprise a plurality of elongated linear transducers. Shown in FIGS. 6A and 6B are two transducers, but any number of transducers can be used. In the embodiment illustrated in FIGS. 6A and 6B transducers 1 are arranged parallel to each other such that the tissue treatment compartment (not specifically designated, see FIG. 2A) is in the volume between them. Thus, in this embodiment, the compartment typically has a rectangular shape. The suction port (not specifically show, see FIG. 2A) can be positioned on the top and/or sides of the compartment. A typical distance between transducers 1 is from about 8 mm to about 12 mm, a typical width of transducers 1 is from about 8 mm to about 12 mm, and a typical height of transducers 1 is from about 1 mm to about 3 mm. A typical length of transducers 1 is from about 10 mm to about 40 mm. Tissue 7 is sucked by vacuum into the compartment between the transducers.

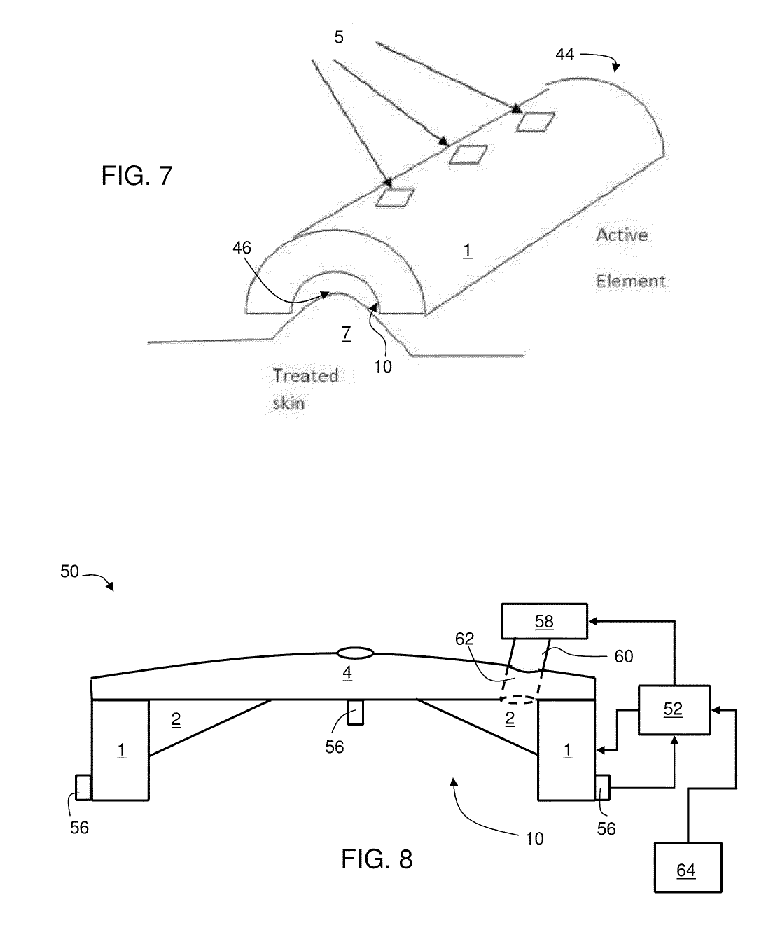

FIG. 7 is a schematic illustration of transducer 1 in embodiment of the invention in which transducer 1 is shaped as half a cylinder.

The term “half-cylinder” in this context does not necessarily mean a half cylinder that covers a spatial angle of 180 degrees. The term half-cylinder of the present embodiments can be any hollow body having a generally round cross-section that covers an angle of between 120 and 200 degrees. The cross-section be circular but may also be non-circular, e.g., elliptic or non-regular.

When transducer 1 is shaped as half a cylinder, tissue treatment compartment 10 is within the volume enclosed by the half-cylinder and therefore has an elongated shape. In some embodiments of the present invention a matching layer (not shown) is introduced between transducer 1 and tissue 7. The matching layer 2 can be provided in a gel form directly applied onto the internal surface of transducer 1 or tissue 7, or it can be provided as a self-supporting layer, for example, a gel enclosed in an ultrasound transparent balloon (e.g., a latex rubber balloon). Several suction ports 5 are preferably distributed on the wall of transducer 1, for example, parallel to the longitudinal axis of the half-cylinder. Alternatively, the front 44 and rear 46 sides of the half cylinder can sealed with one or more suction ports formed in the sealing. When a gel enclosed in a balloon is used, the balloon itself can provide the sealing.

In various exemplary embodiments of the invention device 50 comprises a controller 52 which controls and/or monitors the operation of device 50 and ensures that one or more of the parameters characterizing the treatment are within a predetermined range. For clarity of presentation, controller 52 is not illustrated in FIGS. 2B-7, but the skilled person, provided with the details described herein, would know how to adjust the other drawings to include controller 52.

The parameters controlled by controller 52 include, without limitation, the time (t) of treatment, the frequency (F) of the ultrasound radiation, the power (P) of the ultrasound radiation, the Intensity (I) of the ultrasound irradiation and the Vacuum level within compartment 10. The parameters monitored by controller 52 include, without limitation, the temperature (T) of the tissue, and the depth (D) at which the focal spot is formed (see FIG. 2A).

Controller 52 can receive data pertaining to the above parameters from external sources or, optionally and preferably, from one or more sensors 56. This embodiment is illustrated in FIG. 8. Sensors 56 can provide signals indicative of, for example, temperature, ultrasound frequency, ultrasound power, ultrasound intensity, ultrasound power and pressure within compartment 10. Optionally, controller 52 comprises and/or being associated with a clock for determining the time of treatment.

Controller 52 processes the data, determine the value of the respective parameters and controls the operation of device 50, particularly transducer 1, based on these parameters. Preferably, controller 52 compare each parameter to a predetermine range and controls transducer 1 based on this comparison.

For example, controller 52 can be configured to operate transducer 1 such that the temperature at the focal spot in the dermis is from 60° C. to about 90° C. for a duration of from about 2 to about 60 seconds (See for example Ultrasound and electrotherapy, Applications Techniques & Technology for medical Aesthetics 2002 by Alan Bunting and Laura L root, Chapter 14). Preferably, controller 52 operates transducer 1 such that the temperature of the epidermis is below 50° C. in order to minimize the risk of blistering.

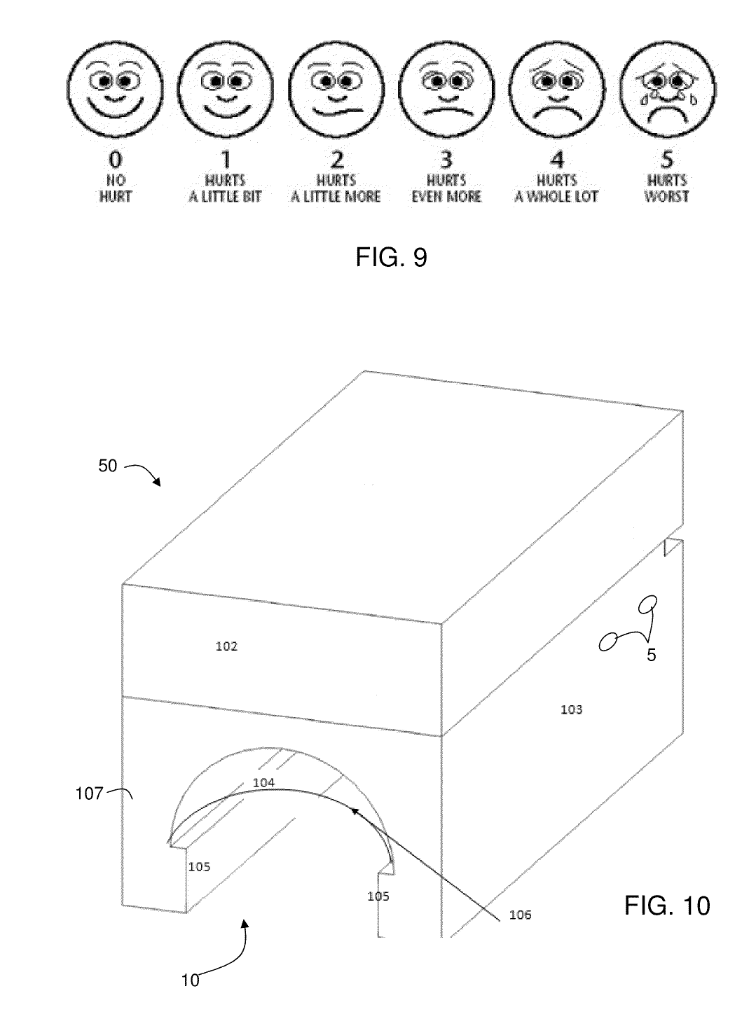

In various exemplary embodiments of the invention controller 52 is configured to operate device 50 according to a predetermined pain scenario. In these embodiments, controller 52 assesses the pain level associated with the monitored set of parameters according to a predetermined pain scale. For example, in some embodiments, the Wong-Baker face rating scale (see FIG. 9) is employed. This scale consists of six faces arranged in a horizontal format and ranging from happy to sad. Each face is associated with a given pain rating as follows. At the far left of the scale, a smiley face indicates a pain rating of zero (0) or “No Hurt”; the next face to the right, which has a slightly less exaggerated smile, indicates a pain rating of two (2) or “Hurts Little Bit”. Moving down the scale, the next face, which is neither smiling nor frowning, indicates a pain rating of four (4) or “Hurts Little More”. The next face is slightly frowning and indicates a pain rating of six (6) or “Hurts Even More”. Continuing down the row, the next face is a more exaggerated frown and indicates a pain rating of eight (8) or “Hurts Whole Lot”. The last face in the series is a crying and frowning face that indicates a pain level of ten (10) or “Hurts Worst”. Other pain scales can also be employed.

Controller 52 can access a database in which each pain level (e.g., each of the six pain levels of the Wong-Baker scale) is associated with a set of parameter ranges. Parameters which may affect the pain level include, without limitation, the temperature, the time of treatment, the depth of focal spot, and the volume of the compartment. Other parameters can also affect the pain level. A pain level database can be prepared, for example, using a research group of volunteers which can evaluate the pain perceived during various treatment scenarios. Based on the entries in the pain-level database, controller 52 can and assess the pain which is currently perceived by the treated subject, and determine whether or not to change one or more parameters or to stop the treatment.

In various exemplary embodiments of the invention device 50 comprises or is associated with a feeding device 58 configured for feeing the anterior of compartment 10 with an acoustic matching material, such as, but not limited to, acoustic gel or the like. Feeding device 58 can deliver the acoustic matching material into the anterior of compartment 10, e.g., via a conduit 60. Fluid communication between conduit 60 and compartment 10 can be established, for example, via an acoustic matching material inlet 62 formed in member 4.

The matching material can delivered at a predetermined rate or adaptively. Also contemplated, are embodiments in which the amount of matching material that is delivered is selected so as to control the free volume within compartment 10. This is useful, for example, when it is desired to control the volume of the tissue within the compartment, wherein larger amount of matching material correspond to smaller volume of tissue in the treatment compartment. This also provides control over the depth of the focal spot as further detailed hereinabove. In various exemplary embodiments of the invention feeding device 58 is controllable by controller 52. Thus, controller 52 can select the amount of matching material depending on the desired depth of focal point.

In some embodiments of the present invention controller 52 is utilized according to a pay-per-use protocol to purchase amounts of operational time for operating device 50. For example, a user or administrator may purchase 100 minutes of operational time of device 50. Once purchased, device 50 may operate for the purchased 100 minutes, including the use of the several session types available. Further, the user or administrator may continue to purchase operational time of device 50 as continued usage is desired. Generally, when the purchase time has elapsed, device 50 ceases to operate until more operational time is purchased. This pay-per-use approach may allow the user or administrator to more accurately control the usage and cost of device 50. In embodiments in which a pay-per-use protocol is employed, device 50 preferably comprises a pay-per-use interface 64 which communicates with controller 52. Interface 64 can be of any known type, including, without limitation, a magnetic stripe reader, smart card reader, keypad or keyboard, coin and bill slots, a biometric input device and the like. For example, for payment, a user may swipe a credit card and enter a billing zip code; a user may swipe a bank card or debit card and enter a password; a user may use a smart card reader and enter a password, or other secure stored value or credit card or token; a user may input paper or coin currency or the like.

FIG. 10 is a schematic illustration of device 50 in embodiments in which device 50 comprises an acoustic waveguide designed and constructed for delivering acoustic energy the sides to the treated tissue.

In these embodiments, device 50 comprises an ultrasound transducer 102, which can be of any type, including any of the ultrasound transducers listed above with respect to transducer 1. In the present example, transducer 102 has a rectangular shape but it can also have other shapes. In the presently preferred embodiment of the invention, device 50 comprises an acoustic waveguide 103 which is in physical contact with transducer 102. Waveguide 103 can comprise any material suitable for guiding mechanical waves. A representative example includes, without limitation, aluminum. The center of acoustic waveguide 103 is made hollow thus defining compartment 10 at the hollow part of waveguide 103. One or more suction ports 5 can be formed on the sides of waveguide 103 for generating under-pressure in compartment 10, as further detailed hereinabove.

Waveguide 103 optionally and preferably comprises a pair of lips 105 protruding inwardly into compartment 10 from the wall 107 of waveguide 103. In various exemplary embodiments of the invention ultrasound energy is delivered to the tissue via contact lips 105 but not via any other part of device 50. Thus, when the tissue (not shown) is sucked into compartment 10 the tissue is devoid of acoustic coupling with any part of waveguide 103 other than lips 105. This configuration ensures that the ultrasound is transmitted only to the sides of the tissue thus preventing or reducing propagation of ultrasound waves deep into the tissue. In some embodiments of the present invention a matching layer (not shown) is placed between lips 105 the tissue for better acoustic coupling.

In some embodiments, device 50 comprises a mechanical barrier 106 disposed within the hollow part of waveguide 103 for preventing the tissue from entering the upper part 104 of waveguide 103. The vertical position of barrier 106 can be made adjustable so as to control the volume of tissue that is sucked into compartment 10 hence also the depth of the focal spot as further detailed hereinabove. Barrier 106 may be formed with holes which are sufficiently large to allow penetration of air and gel through them, and sufficiently small to block the tissue.

In various exemplary embodiments of the invention the distance between contact lips 105 is preferably selected such that a standing ultrasound wave is between the lips. Thus, the distance between contact lips 105 can be integer multiplication of half the wavelength of the ultrasound. The standing wave results in a generally static focal spot at the center of waveguide 103.

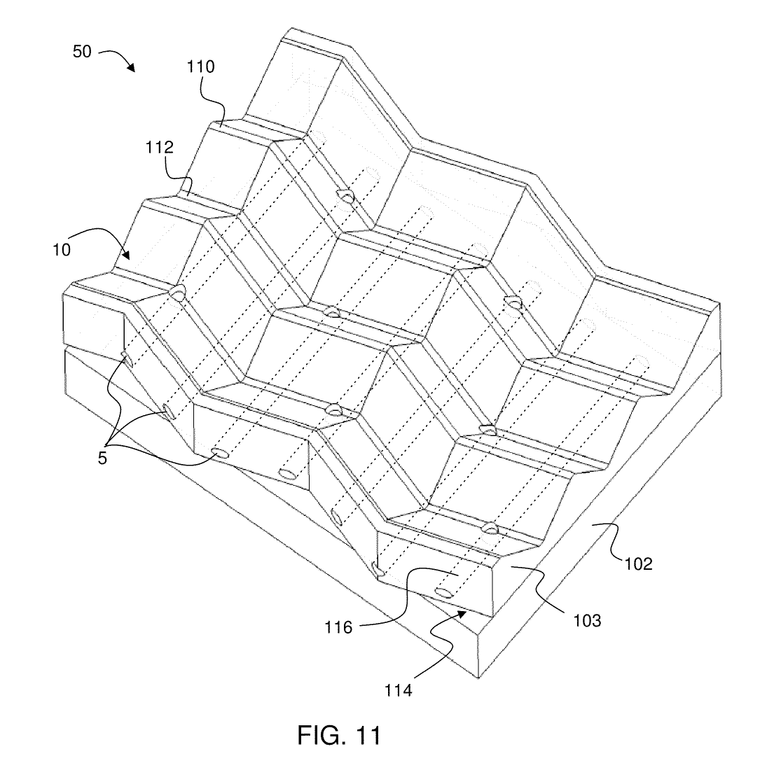

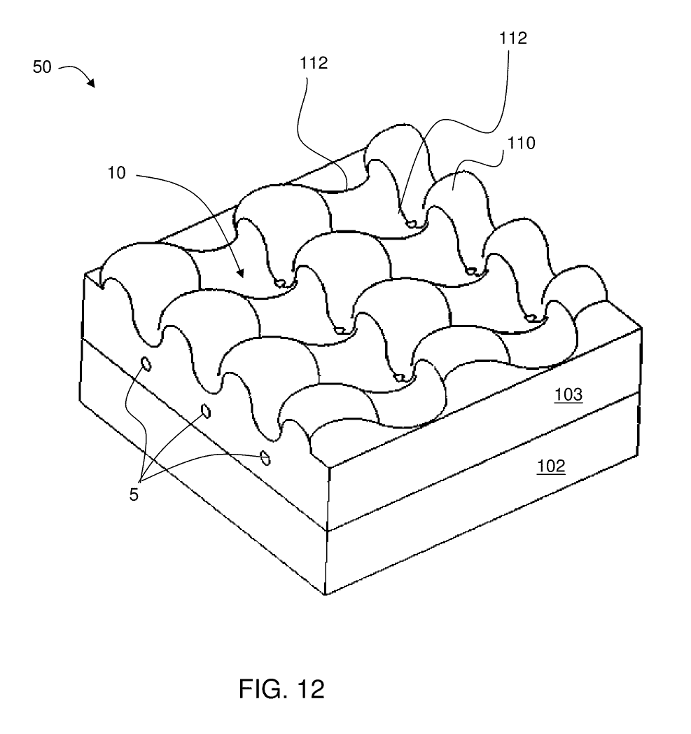

FIGS. 11 and 12 are schematic illustrations of device 50 in embodiments in which the acoustic waveguide has a wavy structure. In these embodiments, acoustic waveguide 103 comprises an alternating pattern of ridges 110 and troughs 112 which form the wavy structure. The troughs can be arranged along one direction, as illustrated in FIG. 11, or along two generally perpendicular directions (e.g., within ±10° deviation), as illustrated in FIG. 12. The volume within each trough 112 defines a tissue treatment compartment 10. The opposite side 114 of the pattern of waveguide 103 is in physical contact with transducer 102. Side 114 can be made planar.

In various exemplary embodiments of the invention several pipes 116 are disposed within waveguide 103. The pipes terminate at the side wall 118 of waveguide 103 to form openings which can serve as suction ports 5. Each pipe is in fluid communication with one or more suction opening 120 form at troughs 112. When an under-pressure is formed in suction ports 5 (for example, via vacuum tubes, see FIG. 2A), the tissue is sucked to occupy troughs 112.

The width of the troughs (distance between adjacent ridges) is preferably selected so as to establish a standing wave within the troughs hence to generate a focal line within each trough.

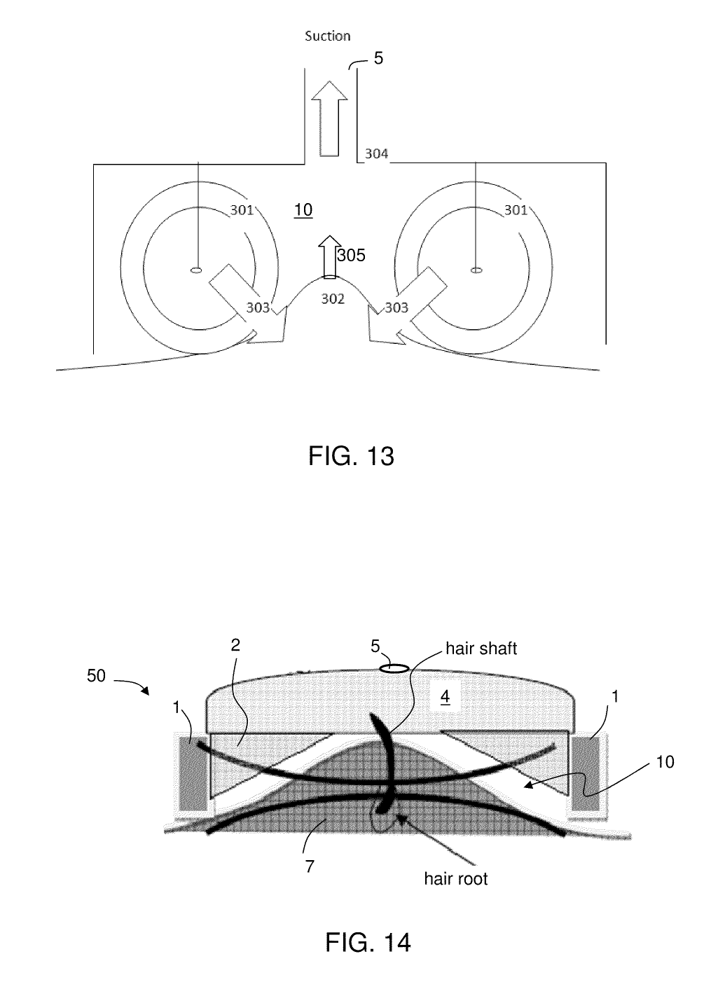

FIG. 13 is a schematic illustration of device 50 in embodiments of the invention in which cylindrical transducers are employed. In these embodiments, device 50 comprises a device body 304 open from one side and having therein a pair of ultrasound transducers 301 each being shaped as a cylinder. Transducers 301 can be made of any material including any material described above in connection to transducer 1. Transducers 301 are arranged within device body 304 such that their symmetry axes are parallel to each other. Preferably, transducers 301 are spaced apart. In various exemplary embodiments of the invention transducers 301 are operative to roll over the skin when device 50 moves from one location to the other. The volume between transducers 301 serves as tissue treatment compartment 10. Device body 304 is formed with one or more suction ports 5 for generating under-pressure within body 304. In use, the open side of device body 304 is placed on a tissue 302 and vacuum is applied through suction port 5, for example, using a vacuum tube (not shown). Tissue 302 is sucked into the volume between transducers 301 as indicated by block arrow 305. Upon activation of transducers 301, ultrasound waves are transmitted from transducer to the tissue at the contact area between the tissue and the transducers, and propagate along directions generally shown by block arrows 303. This creates a focal spot within tissue 302. Once the treatment is completed, the suction is optionally and preferably ceased and device 50 can be relocated while maintaining contact between transducers 301 and the tissue. During the relocation, transducers 301 optionally and preferably roll over the skin thus also adding a massage effect to the treatment.

In any of the embodiments described above, a single device or a plurality of devices can be employed. Single device is preferred from the standpoint of cost and simplicity of operation, and a plurality of devices are preferred from the standpoint of larger area coverage. When a plurality of devices is employed, they can be physically connected to each other such that they can be moved together as a single unit from one location to the other. The devices can be arranged in any arrangement. Representative examples including, without limitation, linear arrangement, two-dimensional arrangement over a Cartesian grid, hive arrangement and the like. The devices can be activated synchronously (e.g., simultaneously, sequentially, alternately) or independently as desired.

The device and method of the present embodiments are particularly useful in stimulating superficial layers of the dermis and hence increasing collagen growth without damaging the epidermis. Hence, the effect of skin rejuvenation is achieved by using ultrasound to stimulate collagen growth substantially without impairing the epidermis. The effect of wrinkle reduction is achieved by using the ultrasound to increase collagen levels in skin without substantially without impairing the epidermis.

The device and method of the present embodiments heat the dermis to some extent, mainly causing collagen denaturation and the following process of collagen regeneration. The device and method of the present embodiments take in, by suction, the treated tissue into the tissue treatment compartment and apply the ultrasound radiation generally parallel (e.g., within ±10° deviation) to the skin surface before suctioning (namely generally parallel to surface 40). This allows safe exposure with small or no penetration to deeper tissue.

The technique of the present embodiments differs from conventional technique in that the ultrasound radiation is generally parallel (e.g., within ±10° deviation) to the surface of the skin (before suction), whereas in conventional techniques the ultrasound radiation is applied perpendicular to the skin surface.

The ultrasound energy of the present embodiments is delivered to a very superficial location. The amount of ultrasound to which non-treated tissue is exposed is small and preferably non-damaging, unlike conventional techniques in which the ultrasound continues to deeper tissue.

The method and device of the present embodiments are also useful for removing hair or destroying the hair roots. These embodiments are illustrated in FIG. 14. Device 50 is placed on the surface of the skin at a location in which the skin has one or more hair roots (including the follicle, dermal papilla, hair bulge and/or a germinal matrix). Vacuum is applied through suction port 5 and the tissue 7 which includes the hair root(s) is sucked into compartment 10. Transducer 1 is activated and ultrasound radiation is delivered into tissue 7. A focal spot is generated at the hair root such that a sufficient amount of heat is generated at the follicle, dermal papilla, hair bulge and/or germinal matrix of the hair. The heat by itself is preferably sufficient to destroy the follicle, dermal papilla, hair bulge and/or germinal matrix.

As used herein, “destruction” refers to hyperthermia, necrosis and/or any other damage to the cells in the follicle (dermal papilla, hair bulge, germinal matrix, etc.) which, at least temporarily, preferably permanently prevents the re-growth of the hair. The prevention of re-growth is preferably for a prolonged period of time, for example, for more than a month, preferably for more than six months, more preferably for more than a year, most preferably permanently.

The method and device of the present embodiments are also useful for cleaning and/or sanitizing wounds. In these embodiments, the device is placed on the surface of the skin at a location in which the skin has one or more wounds. Vacuum is applied through the suction port and the tissue which includes the wound(s) is sucked into the compartment. The ultrasound transducer is activated and ultrasound radiation is delivered into the tissue. It was found by the present inventors that such operation can prevent or reduce infection. The procedure is optionally accompanied by pharmaceutical treatment, such as use of a dermatological composition or the like.

The method and device of the present embodiments are also useful for treating acne. In these embodiments, the device is placed on the surface of the skin at a location in which the skin has one or more acnes. Vacuum is applied through the suction port and the tissue which includes the acne(s) is sucked into the compartment. The ultrasound transducer is activated and ultrasound radiation is delivered into the tissue. It was found by the present inventors that since the acne infections have different percentage of water compared to the surrounding tissue, the ultrasound radiation can selectively treat the acne. The advantage of the device of the present embodiments is the ability to effectively treat a selected depth. For acne treatment, the ultrasound radiation is preferably directed so as to temporarily or permanently destroy the sebaceous glands. The depth at which these glands occur is approximately 1-7 mm, depending on skin thickness and body site. Thus, in these embodiments the focal spot is preferably generated at a depth of from about 1 mm to about 7 mm. The procedure is optionally accompanied by pharmaceutical treatment, such as use of a dermatological composition or via oral delivery.

The method and device of the present embodiments are also useful for stimulation treatment and/or therapeutic effect utilizing a stimulation of acupuncture points. The effectiveness of acupuncture for various illnesses has been recognized for a long time, and has been proven recently in several studies. Traditionally, specific points in the body, called acupuncture points, were reached by piercing with needles for stimulation. The stimulation can also occur from thermal energy by burning Moxa during treatment, or by manual pressure from fingertips or knuckles (acupressure). According to some embodiments of the present invention a device such as device 50 is placed on the surface of the skin at a location in which there is one or more acupuncture points. Vacuum is applied through the suction port and the tissue which includes the acne(s) is sucked into the compartment. The ultrasound transducer is activated and ultrasound radiation is delivered into the tissue so as to generate ultrasound focal spots at the acupuncture points.

The method and device of the present embodiments are also useful for treating blood vessels. For example, the method and device can be used for closing blood vessels by heat. In these embodiments, the device is placed on the surface of the skin and vacuum is applied through the suction port to suck the tissue into the compartment. The ultrasound transducer is activated and ultrasound radiation is delivered into the tissue. The location of the focal spot is selected so as to generate heat at the respective blood vessel. The heat is preferably sufficient to cause vessel closure or sealing. These embodiments are particularly useful for treating varicose veins or spider veins, but other types of blood vessels are not excluded from the scope of the present invention.

The method and device of the present embodiments are also useful for removing tattoo. In these embodiments, the device is placed on the surface of the skin at a location in which the skin has one or more tattoos. Vacuum is applied through the suction port and the tissue which includes the tattoo(s) is sucked into the compartment. The ultrasound transducer is activated and ultrasound radiation is delivered into the tissue. It was found by the present inventors that the ultrasound can be targeted at the ink particles residing in the skin as part of a tattoo thus helping erasing the tattoo.

Additional types of treatments contemplated using the device and method of the present embodiments include, without limitation, preventing infections, stimulating fibroblast cells to activate wound healing, increasing the production of adenosine triphosphate (ATP) for inducing tissue healing, treating sweat glands (e.g., for preventing or reducing sweat in case of over sweating, or for opening closed glands), inducing hair growth or thickening or reducing hair loss, skin cleansing, generating micro massage effect, heating for increasing local blood flow, microdermabrasion, treatment of pressure sores and the like.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition (e.g., dermatological condition), substantially ameliorating clinical or aesthetical symptoms of a condition or substantially preventing the appearance of clinical or aesthetical symptoms of a condition. Representative examples of treatment effects include, without limitation, skin rejuvenator, hair removal, and reduction and prevention of skin wrinkles.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A device for treating a skin tissue, comprising a device body having an anterior compartment and being formed with at least one suction port, and an ultrasound unit configured for generating an ultrasound wave into said anterior compartment;
wherein said device body and said ultrasound unit are designed and constructed such that when an under pressure is formed in said suction port, the skin tissue is drawn by vacuum into said anterior compartment and irradiated by said ultrasound wave to form a focal spot at a distance of from about 0.2 mm to about 4 mm below an external surface of the skin tissue.

2. The device of claim 1, wherein said ultrasound unit comprises an annular ultrasound transducer and wherein at least part of said device body is defined by said annular ultrasound transducer.

3. The device of claim 2, wherein said annular ultrasound transducer has a shape selected from the group consisting of a ring, a truncated cone and a spherical segment.

4. The device of claim 1, wherein said ultrasound unit comprises a plurality of elongated linear ultrasound transducers configured to generate ultrasound waves in a plurality of directions being generally parallel to an external skin surface outside said anterior compartment and adjacent thereto.

5. The device of claim 1, wherein said ultrasound unit comprises a pair of elongated linear ultrasound transducers configured to generate ultrasound waves in opposite and co-linear directions being generally parallel to an external skin surface outside said anterior compartment and adjacent thereto.

6. The device of claim 1, wherein said ultrasound unit comprises an ultrasound transducer shaped as a generally half-cylinder, wherein said at least one suction port is formed at a wall of said transducer generally along an apex of said half-cylinder.

7. The device of claim 1, wherein said acoustic unit comprises an acoustic waveguide defining said anterior compartment and an ultrasound transducer being acoustically coupled to said acoustic waveguide such that said acoustic wave is transmitted from said ultrasound transducer to said and from said acoustic waveguide to the skin tissue.

8. The device of claim 7, wherein said acoustic waveguide comprises a pair of contact lips opposite to each other, said contact lips being configured for contacting the skin tissue such that ultrasound energy is delivered to the skin tissue via said contact lips but not via any other part of the device.

9. The device of claim 8, wherein a distance between said contact lips is selected such as to form a standing wave therebetween.

10. The device of claim 7, wherein said acoustic waveguide has a wavy structure having ridges and troughs and wherein said anterior compartment is defined within said troughs.

11. The device of claim 10, wherein said acoustic waveguide is formed with at least one pipe therein having a first end at a trough of said wavy structure and a second end at a wall of said wavy structure to form said at least one suction port.

12. The device of claim 1, wherein said acoustic unit comprises a plurality of cylindrical ultrasound transducers, and wherein said anterior compartment is defined between two adjacent transducers.

13. The device of claim 12, wherein said cylindrical ultrasound transducers are arranged within said device body and wherein said suction port is formed at a wall of said device body.

14. The device of claim 12, wherein said cylindrical ultrasound transducers are operative to roll on a surface of the skin tissue.

15. The device of claim 1, wherein said acoustic unit comprises an ultrasound transducer having an ultrasound emitting surface which is slanted at an angle with respect to an external skin surface outside said anterior compartment and adjacent thereto.

16. The device of claim 1, further comprising an ultrasound matching layer at least partially occupying said anterior compartment and being selected for enhancing acoustic coupling between said ultrasound wave and the skin tissue.

17. The device of claim 16, wherein said ultrasound matching layer is a gel being in direct contact with an internal wall of said anterior compartment.

18. The device of claim 16, wherein said ultrasound matching layer is self-supporting layer.

19. The device of claim 1, further comprising a top member at which said suction port is formed, wherein said top member is characterized ultrasound reflectivity of less that 50%.

20. The device of claim 1, further comprising a suction device being an fluid communication with said suction port and configured for generating said under pressure.

21. The device of claim 1, further comprising a cooling conduit configured for redirecting air sucked from said anterior compartment onto said acoustic unit so as to cool said acoustic unit.

22. The device of claim 1, further comprising a controller configured for controlling and/or monitoring at least one parameter, selected from the group consisting of time of treatment, frequency of said ultrasound wave, power of said ultrasound wave, intensity said ultrasound wave, vacuum level within said anterior compartment, temperature of the skin tissue, and depth of said focal spot.

23. The device of claim 22, wherein said controller is configured for controlling said vacuum level so as to adjust said depth of said focal spot.

24. The device of claim 22, further comprising at least one sensor for providing said controller with data pertaining to at least one of said parameters.

25. The device of claim 1, further comprising a feeding device configured for feeing said anterior compartment with an acoustic matching material.

26. The device of claim 25, further comprising a controller configured for controlling said feeding device so as to select a volume of said acoustic matching material hence to control a free volume within said anterior compartment hence also to adjust a depth of said focal spot.

27. The device of claim 22, wherein said controller is configured for accessing a pain-level database and comparing at least one said parameters to parameters in said pain-level database so as to assess a pain level associated with the skin tissue treatment.

28. The device of claim 22, further comprising a pay-per-use interface communicating with said controller.

29. A method of treating a skin tissue, comprising drawing the skin tissue by vacuum into an anterior compartment of a device body and generating an ultrasound wave into said anterior compartment, such as to form a focal spot at a distance of from about 0.2 mm to about 4 mm below an external surface of the skin tissue.

30. The method of claim 29, wherein said focal spot is formed in the dermis of the skin tissue but not in the epidermis thereof.

31. The method of claim 29, wherein said focal spot is formed in the dermis of the skin tissue but not in the hypodermis thereof.

32. The method of claim 29, wherein said ultrasound wave is generated along a direction being generally parallel to an external skin surface outside said anterior compartment and adjacent thereto.

33. The method of claim 29, wherein said generating said ultrasound wave is in a direction which is slanted at an angle with respect to an external skin surface outside said anterior compartment and adjacent thereto.

34. The method of claim 29, wherein said ultrasound wave is transmitted only to the sides of said drawn skin tissue.

35. The method of claim 29, wherein said generating said ultrasound wave is by a plurality of rotatable cylindrical ultrasound transducers, and wherein the method comprises rolling said cylindrical ultrasound transducers on the surface of the skin tissue.

36. The method of claim 29, further comprising reducing reflection of said ultrasound wave from a top part of said anterior compartment.

37. The method of claim 29, further comprising adjusting vacuum level in said anterior compartment so as to adjust said depth of said focal spot.

38. The method of claim 29, further comprising feeding acoustic matching material into said anterior compartment, wherein said an amount of said matching material is selected so as to adjust a depth of said focal spot.

39. The method of claim 29, further comprising assessing a pain level associated with the skin tissue treatment.

40. The method of claim 29, wherein said treating comprises at least one treatment selected from the group consisting of: increasing collagen growth, hair removal, hair root destruction, wound cleaning, wound sanitizing, destruction of acne, acupuncture points stimulation, blood vessels treatment, tattoo removal, infection prevention, fibroblast cells stimulation, increasing the production of adenosine triphosphate (ATP), sweat glands treatment, skin cleansing, massaging, increasing local blood flow, microdermabrasion, and pressure sores treatment.
